(19) United States
(12) Patent Application Publication
(10) Pub. No.: US 2003/0199759 A1
(43) Pub. Date: Oct. 23, 2003

(54) Coronary catheter with radiopaque length markers

(76) Inventor: Merwin F. Richard, Yonkers, NY (US)

Correspondence Address: Bruce H. Johnsonbaugh, Eckhoff & Hoppe, Suite 3125, 333 Market Street, San Francisco, CA, 94105, US

(21) Appl. No.: 10/417,474

(22) Filed: Apr. 15, 2003

Related U.S. Application Data
(60) Provisional application No. 60/373,593, filed on Apr. 18, 2002.

(51) Int. Cl. A61B 5/05

(52) U.S. Cl. 600/426

(57) ABSTRACT

A coronary catheter is provided having a series of radiopaque length markers applied to the mid-region of the catheter, wherein the mid-region of the catheter lies directly behind the heart when the catheter is in its deployed position prior to coronary angioplasty. The length markers are applied to the mid-region a known distance apart, allowing the length of a coronary occlusion to be directly measured on an x-ray image of the occlusion by simply comparing the length of the occlusion with the known distance between two or more of the radiopaque markers.

### CROSS-REFERENCE TO RELATE APPLICATION

This application claims the benefit of and priority from U.S. provisional application Serial No. 60/373,593 filed Apr. 18, 2002.

### BACKGROUND AND BRIEF SUMMARY OF INVENTION

This invention pertains generally to coronary catheters. More particularly, the present invention provides radiopaque length markers along the central region of the catheter which extends behind the patient's heart. The radiopaque length markers applied on this portion of the catheter facilitates fairly accurate direct measurement of the length of coronary artery occlusions so that a stent of proper length can be utilized in the affected coronary artery.

The problem addressed by the present invention is accurately and quickly determining the length of a coronary artery occlusion during a balloon angioplasty/stenting surgical procedure. Knowing the precise length of the occlusion allows the cardiologist to utilize a stent of proper length. Using a stent of improper length may have serious consequences for the patient, or at the very least require removal of the improper stent and a second procedure. The use of a stent which is too short results in restinosis. A stent which is too long may cause rupturing of the artery. At present, the cardiologist examines an x-ray taken during the surgical procedure. Unfortunately, the x-ray has no direct reference measurement points or markers. The cardiologist must estimate the length of stent to be used. The present invention solves this problem by applying radiopaque length markings to the catheter used in the procedure. The present invention thereby allows the cardiologist to quickly make a relatively precise, direct measurement of the occlusion length based on the x-ray. The direct measurement afforded is significantly more accurate than the estimates required by the prior art.

It is known in the prior art to apply radiopaque markings at the extreme distal end of a catheter as shown in U.S. Pat. Nos. 6,285,903 and 4,279,252. Those markers are used for measuring distance within chambers in the heart and for judging the rotational attitude of the tip of the catheter. It is also known in the prior art to apply radiopaque markings to guide wires as shown in U.S. Pat. No. 6,179,788.

The present invention is believed to be the first occasion in which radiopaque length markings are applied to the mid-region of the catheter rather than at its extreme distal tip. By applying the radiopaque length markers to the mid-region of the catheter which lies behind the patient's heart when the catheter is in its deployed position prior to coronary angioplasty, direct length measurements can be made of coronary artery occlusions.

A primary object of the invention is to provide an apparatus which increases the accuracy of measurement of coronary occlusions to assure that the proper length stent is used to treat the occlusion.

A further object of the invention is to provide an apparatus which provides a cardiologist with a catheter having reference length markers to facilitate quick, direct measurement of the length of coronary occlusions.

Other objects will become apparent from the following description and drawings, wherein:

### BRIEF DESCRIPTION OF THE DRAWINGS

FIG. 1 is a schematic representation of a patient on a standard cardiovascular examination table undergoing x-ray examination;

FIG. 2 is a schematic representation of the patient shown in FIG. 1 as the x-ray equipment is rotated around the patient; and

FIG. 3 is a schematic representation of the present invention fully deployed inside the patient's heart and aorta.

### DETAILED DESCRIPTION OF THE DRAWINGS

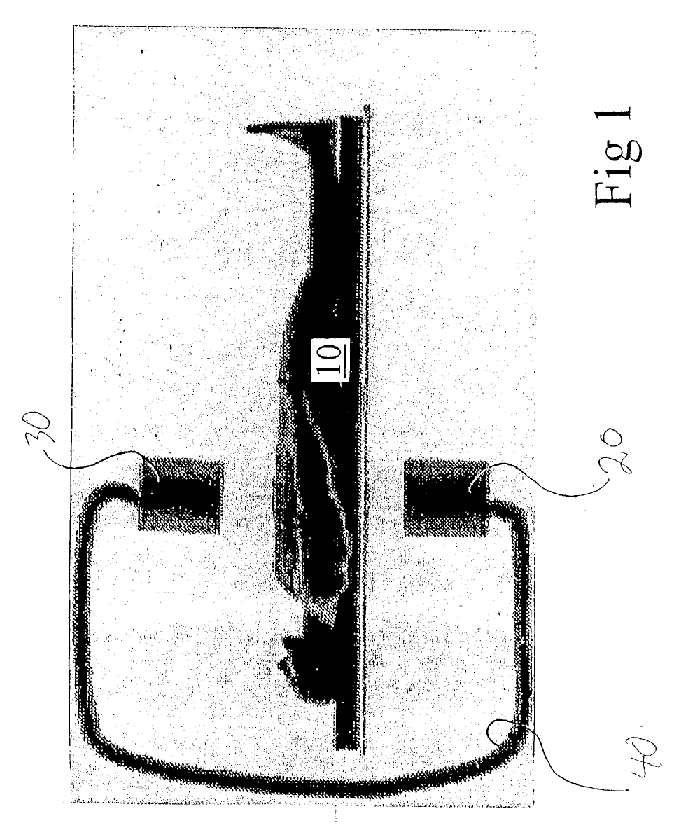

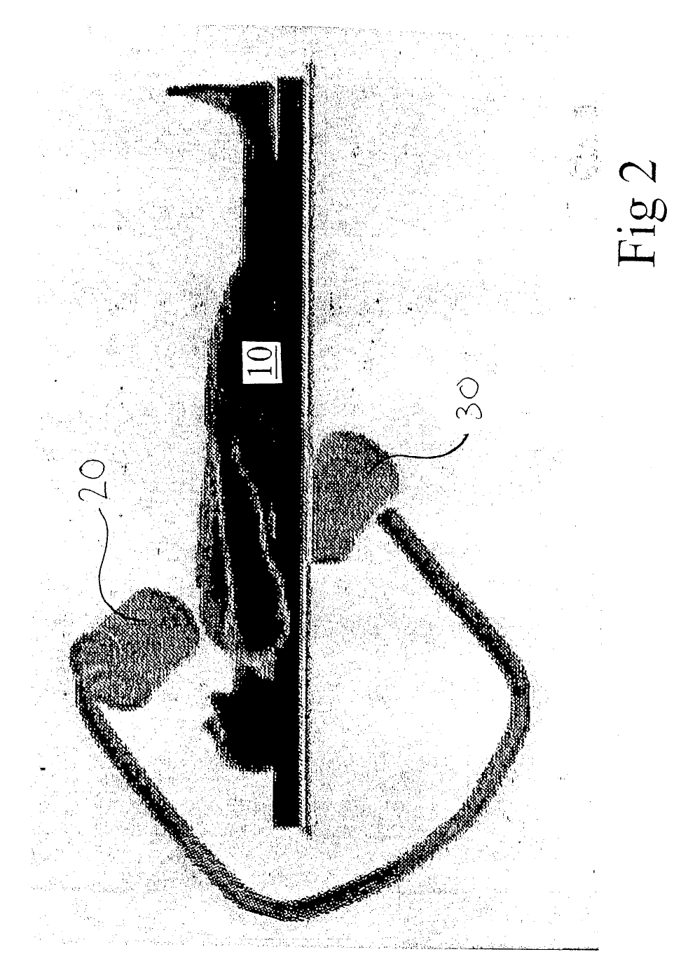

FIG. 1 illustrates a patient on a standard cardiovascular examination table. An x-ray emitter 20 is shown directly beneath the patient and an image intensifier 30 shown directly above the patient 10. The x-ray emitter 20 and imagine intensifier 30 are joined by a rotatable connector 40 which allows the assembly to rotate a full 360° around the patient as shown best in FIG. 2 wherein the x-ray emitter 20 is shown slightly above the patient and the image intensifier 30 is shown slightly below the patient.

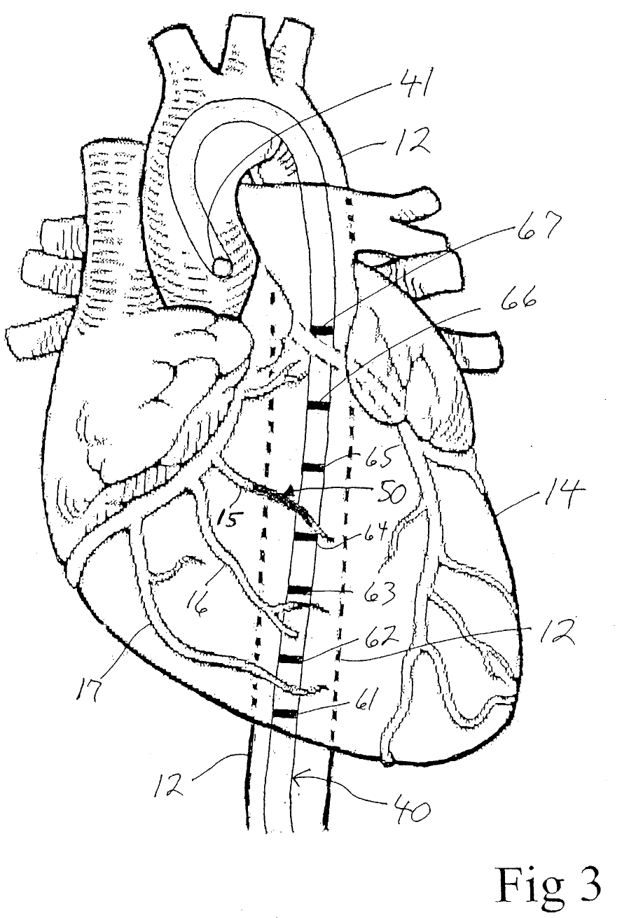

FIG. 3 illustrates catheter 40′ according to the present invention fully deployed in aorta 12 with its distal end 41 inside the patient's heart 14. The portion of aorta 12 that extends behind the heart 14, as shown in FIG. 3, is illustrated in phantom. Once the catheter 40 is deployed, as shown in FIG. 3, and prior to insertion of a guide wire and balloon/stent assembly, dye is injected through catheter 40 and through distal end 41 to illuminate any occlusions in coronary arteries 15, 16, 17, for example. Only a portion of the coronary arteries is numbered in the interest of clarity. As shown in FIG. 3, coronary artery 15 has an occlusion 50.

According to the present invention, a plurality of radiopaque length markers 61-67 are placed on the surface of catheter 40 along the central region of the catheter, i.e., that portion of catheter 40 which extends directly behind the heart 14 when the catheter is in its deployed position illustrated in FIG. 3. As shown in FIG. 3, the markings 61-67 are placed at one centimeter intervals on the surface of catheter 40.

After a full 360° x-ray examination and before a stenting procedure is initiated, the length of occlusion 50 is measured by simply comparing it either visually or with a measuring device to the closest of the radiopaque markings 61-67. The occlusion 50 in artery 15 is shown at its full length by selecting the image from the 360° scan that shows occlusion 50 at its full length. As shown in FIG. 3, occlusion 50 has a length of approximately one centimeter determined simply by comparing the length of the occlusion 50 to the distance between markings 64 and 65. This length determination of the occlusion 50 allows the cardiologist a high level of confidence in selecting a stent of proper length to treat occlusion 50 with a single procedure.

A significant drawback of prior art stenting procedures is that the cardiologist must extimate the length of the occlusion without having direct reference measurement points in the x-ray that best illuminate the length of the occlusion. The present invention eliminates that uncertainty inherent in the prior art.

It is within the scope of the invention to utilize radiopaque markings of different spacings than that shown in FIG. 3. As shown in FIG. 3, the radiopaque markings are preferably solid lines extending around the circumference of the mid-region of the catheter. Other patterns of markings may be utilized, such as dashed or dotted lines, as long as the markings present a clear image on the x-ray output.

The foregoing description of the invention has been presented for purposes of illustration and description and is not intended to be exhaustive or to limit the invention to the precise form disclosed. Modifications and variations are possible in light of the above teaching. The embodiments were chosen and described to best explain the principles of the invention and its practical application to thereby enable others skilled in the art to best use the invention in various embodiments and with various modifications suited to the particular use contemplated. The scope of the invention is to be defined by the following claims.

What is claimed is:

1. A coronary catheter for use in treatment of an occluded coronary artery with balloon angioplasty and placement of a stent in said occluded artery, wherein dye is injected through said catheter and x-rays are taken to illuminate said occlusion immediately prior to coronary angioplasty, comprising:
an elongated catheter having a distal end and a mid-region, said mid-region lying directly behind the heart when said catheter is in its deployed position prior to coronary angioplasty, and
a plurality of radiopaque length markers applied a known distance apart to said mid-region of said catheter, whereby the length of said coronary occlusion may be directly measured on said x-ray image by comparing the length of said occlusion with the known distance between two or more of said radiopaque markers.

2. The apparatus of claim 1 wherein said markings are solid lines extending around the circumference of said catheter.

3. The apparatus of claim 2 wherein said markings are spaced apart a distance of one centimeter.
